(19) United States
(12) Patent Application Publication
(10) Pub. No.: US 2026/0176679 A1
(43) Pub. Date: Jun. 25, 2026

(54) NOVEL NUCLEIC ACID ISOTHERMAL AMPLIFICATION METHOD USING EXTENSION-MEDIATED SELF- FOLDING

(71) Applicant: KOREA UNIVERSITY RESEARCH AND BUSINESS FOUNDATION, Seoul (KR)

(72) Inventors: Cheul Hee JUNG, Seoul (KR); Ha Rim SAGONG, Seoul (KR)

(21) Appl. No.: 19/127,876

(22) PCT Filed: Nov. 1, 2023
(86) PCT No.: PCT/KR2023/017187

§ 371 Date: Sep. 10, 2025

(30) Foreign Application Priority Data
Nov. 9, 2022 (KR) 10-2022-0148730
Oct. 31, 2023 (KR) 10-2023-0148031

(51) Int. Cl. C12Q 1/6853; C12Q 1/70

(52) U.S. Cl. CPC C12Q 1/6853; C12Q 1/701

(57) ABSTRACT

The present invention relates to a novel nucleic acid isothermal amplification method using extension-mediated self-folding. Specifically, the present invention relates to a primer set for extension-mediated self-folding isothermal amplification, a kit for extension-mediated self-folding isothermal amplification comprising the primer set, a target nucleic acid isothermal amplification method using the primer set for extension-mediated self-folding isothermal amplification, and a target nucleic acid detection method using the primer set for extension-mediated self-folding isothermal amplification, the primer set for extension-mediated self-folding isothermal amplification consisting of a pair of primers, which are a forward primer and a reverse primer, wherein the 3′ region of each primer comprises an annealing site consisting of 15 to 25 base sequences complementary to a target sequence to be detected, the 5′ region of each primer comprises a self-folding site consisting of 10 to 25 base sequences complementary to the nucleic acid sequence of an amplification reaction product so as to form a hairpin structure, and an unnatural base, isodG, is inserted between the annealing site and the self-folding site.

### TECHNICAL FIELD

The present disclosure relates to a novel nucleic acid isothermal amplification method using extension-mediated self-folding.

### BACKGROUND ART

Nucleic acid amplification refers to a method that exponentially amplifies traces of nucleic acid targets by using the characteristics of nucleic acids. Polymerase chain reaction (PCR, hereinafter PCR) is one of the most widely employed among various nucleic acid amplification methods. PCR involves repeated cycles of denaturation, annealing, and extension, which can be achieved by precise temperature control. PCR is sensitive enough to detect even several copies of nucleic acid targets, but has the limitation of requiring a thermocycler capable of accurate temperature control.

To overcome this problem, isothermal amplification technology has been developed that enables amplification at a constant temperature even without the thermocycler. This technology can be attained by using polymerases with strand displacement activity, and a representative isothermal amplification method is loop-mediated isothermal amplification (hereinafter, LAMP), which can detect several dozen copies of nucleic acid targets within tens of minutes. However, LAMP has the drawback of complicated primer design due to the use of 4 to 6 primers. Out of these, inner primers and loop primers contain two target recognition sequences and thus have a long length of approximately 40 nt. The long length of the primers facilitates the formation of non-specific amplification products, such as primer-dimer formation, thereby causing false-positive results. Moreover, most LAMP amplification products are double-stranded DNA (hereinafter, dsDNA), excluding the loop sequence, and thus most of LAMP-based diagnostic methods use non-specific detection strategies, such as turbidity change, color change, or fluorescence detection using intercalating dyes.

To resolve the conventional problems, sequence-specific probes (hereinafter, probes), such as molecular beacon (hereafter, MB) or one-step strand displacement (hereafter, OSD) probes, were developed, and the use of these techniques can specifically detect only target amplification products. However, the single-stranded DNA (hereinafter, ssDNA) is limited to only the loop sequence, and thus designing and applying such probes are also restricted.

Therefore, there is a need for a novel nucleic acid isothermal amplification method capable of resolving the-above described problems.

### DISCLOSURE OF INVENTION

### Technical Problem

While researching to develop techniques that resolve the complexity of primer design and the limitations of probe design, which are the disadvantages of existing LAMP, and allows target-specific detection through isothermal amplification, the present inventors developed a novel nucleic acid isothermal amplification method using extension-mediated self-folding, which is named “extension-mediated self-folding isothermal amplification technology (hereinafter, ExIT)”. The present inventors identified that the developed method enables amplification even with using only self-folding primers (SP) and the production of single-stranded DNA (hereinafter, ssDNA) amplification products through an unnatural base introduced into the self-folding primers, and also identified that the developed method enables target-specific detection and ensures accurate distinction of target amplification products even in the incorporation of non-specific specimens, thereby achieving high detection accuracy and allowing the detection of targets at low concentrations, and thus completed the present disclosure.

Accordingly, an aspect of the present disclosure is to provide a primer set for extension-mediated self-folding isothermal amplification, the primer set including a pair of primers consisting of a forward primer and a reverse primer, wherein the 3′-region of each of the primers includes an annealing site consisting of a 15- to 25-nucleotide sequence complementary to a target sequence of a target subject, the 5′-region of each of the primers includes a self-folding site consisting of a 10- to 25-nucleotide sequence complementary to a nucleic acid sequence of an amplification reaction product to form a hairpin structure, and isodG, an unnatural base, is inserted between the annealing site and the self-folding site.

Another aspect of the present disclosure is to provide a kit for extension-mediated self-folding isothermal amplification to detect a target nucleic acid, the kit including the primer set of the present disclosure.

Still another aspect of the present disclosure is to provide a method for isothermal amplification of a target nucleic acid by using a primer set for extension-mediated self-folding isothermal amplification, the method including subjecting a reaction mixture, containing a target nucleic acid of a target subject and the primer set of the present disclosure, to isothermal amplification.

Still another aspect of the present disclosure is to provide a method for detection of a target nucleic acid by using a primer set for extension-mediated self-folding isothermal amplification, the method including subjecting a specific target nucleic acid, contained in a biological sample, to isothermal amplification by using the primer set of the present disclosure.

### Solution to Problem

In accordance with an aspect of the present disclosure, there is provided a primer set for extension-mediated self-folding isothermal amplification, the primer set including a pair of primers consisting of a forward primer and a reverse primer, wherein the 3′-region of each of the primers includes an annealing site consisting of a 15- to 25-nucleotide sequence complementary to a target sequence of a target subject, the 5′-region of each of the primers includes a self-folding site consisting of a 10- to 25-nucleotide sequence complementary to a nucleic acid sequence of an amplification reaction product to form a hairpin structure, and isodG, an unnatural base, is inserted between the annealing site and the self-folding site.

In an embodiment of the present disclosure, the unnatural base may be one isodG that is inserted.

In an embodiment of the present disclosure, the self-folding site may consist of a 12- to 21-nucleotide sequence complementary to the nucleic acid sequence of the amplification reaction product to form a stable hairpin structure.

In accordance with another aspect of the present disclosure, there is provided a kit for extension-mediated self-folding isothermal amplification to detect a target nucleic acid, the kit including the primer set of the present disclosure.

In an embodiment of the present disclosure, the target nucleic acid may be derived from a virus or a pathogenic microorganism.

In accordance with still another aspect of the present disclosure, there is provided a method for isothermal amplification of a target nucleic acid by using a primer set for extension-mediated self-folding isothermal amplification, the method including subjecting a reaction mixture, containing a target nucleic acid of a target subject and the primer set of the present disclosure, to isothermal amplification.

In an embodiment of the present disclosure, a reaction product in a single-stranded DNA (ssDNA) state may be produced by the isothermal amplification.

In an embodiment of the present disclosure, the isothermal amplification may be performed at a temperature of 60 to 65° C.

In accordance with still another aspect of the present disclosure, there is provided a method for detection of a target nucleic acid by using a primer set for extension-mediated self-folding isothermal amplification, the method including subjecting a specific target nucleic acid, contained in a biological sample, to isothermal amplification by using the primer set of the present disclosure.

In an embodiment of the present disclosure, a target nucleic acid reaction product in a single-stranded DNA (ssDNA) state may be produced by the isothermal amplification.

In an embodiment of the present disclosure, the isothermal amplification may be performed at a temperature of 60 to 65° C.

In an embodiment of the present disclosure, the target nucleic acid may be derived from a virus or a pathogenic microorganism.

### Advantageous Effects of Invention

The present disclosure is directed to a novel primer set for isothermal amplification capable of resolving the problems of existing loop-mediated isothermal amplification (LAMP) and a novel extension-mediated self-folding isothermal amplification technology (ExIT) using the primer set. The method provided in the present disclosure, unlike existing methods, can resolve the complexity of primer design and implement the exponential amplification by using only one pair of primers, and exhibit an effect of producing a large amount of single-stranded amplification product (ssDNA) by inducing self-folding after extension. Therefore, the method for extension-mediated self-folding isothermal amplification using a self-folding primer pair of the present disclosure can detect a target nucleic acid sensitively and accurately, detect low concentrations of targets with high reproducibility and precision, and enables quantitative analysis. This method of the present disclosure can be applied to various isothermal amplification methods and thus can be utilized even for a diagnostic method with a reduced risk of false positive.

### BRIEF DESCRIPTION OF DRAWINGS

FIG. 1 is a schematic diagram illustrating an overall operational mechanism of extension-mediated self-folding Isothermal amplification technology (ExIT) provided in the present disclosure.

FIG. 2 is a schematic diagram illustrating the comparison of self-folding mechanisms for different distances between target recognition sequences.

FIG. 3 is a schematic diagram illustrating the folding efficiency difference according to the presence or absence of the distance between target sequences recognized by self-folding primers.

FIG. 4 illustrates the results of analyzing the self-folding efficiency according to the length of the self-folding site of each primer, as an optimal amplification condition for self-folding primers, under different temperature conditions.

FIG. 5 shows real-time fluorescence curves for the lengths of the folding site of reverse self-folding primes (RSP) of the present disclosure.

FIG. 6 shows real-time fluorescence curves for the lengths of the folding site of forward self-folding primes (FSP) of the present disclosure.

FIG. 7 illustrates the results of investigating the analysis limits for four selected candidate primer sets in an exemplary embodiment of the present disclosure.

FIG. 8 shows schematic diagrams illustrating the comparison of mechanisms of reaction amplification product formation using self-folding primers with and without isodG.

FIG. 9 illustrates the results of analyzing the detection sensitivity of the target nucleic acid (SARS-CoV-2 N gene) by using self-folding primers with or without isodG, and panel A shows the results obtained using primers without isodG, and panel B shows the results obtained using primers having isodG inserted therein.

FIG. 10 illustrates the results of analyzing the relationship between the detection time and the log value of the SARS-CoV-2 concentration (9×10¹ to 9×10⁴ copies).

FIG. 11 shows the results of analyzing the detection specificity of a target nucleic acid (SARS-CoV-2 N gene) through extension-mediated self-folding isothermal amplification technology using self-folding primers of the present disclosure.

### BEST MODE FOR CARRYING OUT THE INVENTION

The present disclosure provides a primer set for extension-medicated self-folding isothermal amplification, capable of amplifying a target nucleic acid without an auxiliary enzyme or an additional primer under isothermal conditions, and a method for extension-medicated self-folding isothermal amplification by using the primer set.

The primer set for extension-mediated self-folding isothermal amplification provided in the present may include a pair of primers consisting of a forward primer and a reverse primer, wherein the 3′-region of each of the primers includes an annealing site consisting of a 15- to 25-nucleotide sequence complementary to a target sequence of a target subject, the 5′-region of each of the primers includes a self-folding site consisting of a 10- to 25-nucleotide sequence complementary to a nucleic acid sequence of an amplification reaction product to form a hairpin structure, and isodG, an unnatural base, is inserted between the annealing site and the self-folding site.

More specifically, the primer set for extension-medicated self-folding isothermal amplification of the present disclosure enables isothermal amplification by only one pair of primers, unlike conventional isothermal amplification requiring 4 to 6 primers.

Additionally, the primers for extension-medicated self-folding isothermal amplification of the present disclosure may form a self-folding structure.

The inner primer of existing LAMP recognizes two target sequences, and the distance between the sequences is about 20 to 40 nt. In order for the inner primer, after extension, to form a hairpin structure and again expose a target recognition sequence, the strand displacement activity of the outer primer is needed due to the extension. However, it was designed in the present disclosure that this target recognition sequence distance is removed to form a folding structure without the help of the outer primer, thereby opening the target recognition sequences.

That is, the 3′-region of each primer of the primer set includes a nucleotide sequence complementary to a target sequence of a target subject, and this nucleotide sequence is named an annealing site.

Specifically, the nucleotide sequence complementary to the target sequence, that is, the annealing site may be designed to be a 15- to 25-nucleotide sequence.

Additionally, the 5′-region of each primer of the primer set includes a nucleotide sequence complementary to a nucleic acid sequence of an amplification reaction product so as to form a hairpin structure, and this nucleotide sequence is named a self-folding site.

Specifically, the folding site is composed of a nucleotide sequence complementary to a nucleic acid sequence of a reaction product, obtained by amplification through isothermal amplification after complementary binding of the annealing site of the primer to a target sequence. The nucleic acid sequence of the amplified reaction product may be a nucleotide sequence located right next to the annealing site, and such a folding site may be designed to be a 10- to 25-nucleotide sequence, and preferably, may be designed to be a 12- to 21-nucleotide sequence.

Particularly, in the designing of the primers for extension-mediated self-folding isothermal amplification of the present disclosure, the length of the folding site of each primer may affect the maintenance of the folding structure after the extension reaction of the primer.

Therefore, it is important to design the folding site to be 10 to 25 nucleotides in length, preferably 12 to 21 nucleotides in length, and the reason is that a too short folding site makes it difficult to maintain the folded structure, while a too long folding site increases the likelihood of primer dimer formation.

Therefore, it is important that the length of the folding site has the above-described length.

Furthermore, in the primer set of the present disclosure, that is, a forward primer and a reverse primer each including an annealing site and a folding site, isodG, an unnatural base, is inserted between the annealing site and the self-folding site.

Particularly, the primer set for extension-medicated self-folding isothermal amplification provided in the present disclosure has isodG, an unnatural base, inserted between the annealing site and the self-folding site, causing a polymerase reaction to stop at the position of isodG, thereby obtaining a reaction amplification product in a single-stranded DNA state. Through such a cycling pathway where an amplification product in an ssDNA state is repeatedly produced, target nucleic acid in an ssDNA state can be obtained.

Conventional LAMP involves amplification through an intermediated product, wherein the 3′-region of the intermediated product is extended and, during the formation of a dsDNA amplification product, inner and loop primers are bound to even the loop site to form a novel reaction amplification product. If this process is repeated, most of complex reaction products mainly consisting of dsDNA are formed, resulting in difficulty in accurate analysis.

However, the present disclosure is directed to a method capable of resolving such problems, wherein isodG was inserted inside the primers, and the amplification reaction was stopped at the position of isodG, thereby preventing the production of undesired reaction products. In addition, the insertion of the unnatural base may indicate that one isodG may be inserted between the annealing site and the folding site.

A reaction product in an ssDNA state can be obtained by inserting isodG between the annealing site and the folding site of the primer (SP) of the present disclosure. If isodG is not contained within the primer, amplification proceeds up to the end of the SP, ultimately resulting in double-stranded amplification products. The double-stranded structure is structurally stable, thereby making it difficult to form a hairpin structure through self-folding and to enable probe binding. However, the insertion of isodG between the annealing site and the folding site enables the extension of polymerase only up to the annealing site, and thus the folding site remains in an ssDNA state, thereby forming a stable folding structure. Furthermore, all the reaction amplification products are formed in a single strand state, thereby making it easier to design and use a probe.

In the present disclosure, the insertion of the unnatural base may indicate that one isodG may be inserted between the annealing site and the folding site.

In the present disclosure, the isodG, which is iso-guanosine, generally refers to an isomer nucleotide for guanine nucleotide introduced into RNA or DNA.

The primers for extension-medicated self-folding isothermal amplification provided in the present disclosure are bound to a target sequence and then form a folding structure by an extension reaction, and the formation of the folding structure exposes the annealing site, thereby inducing the approach and extension of novel primers for extension-medicated self-folding isothermal amplification.

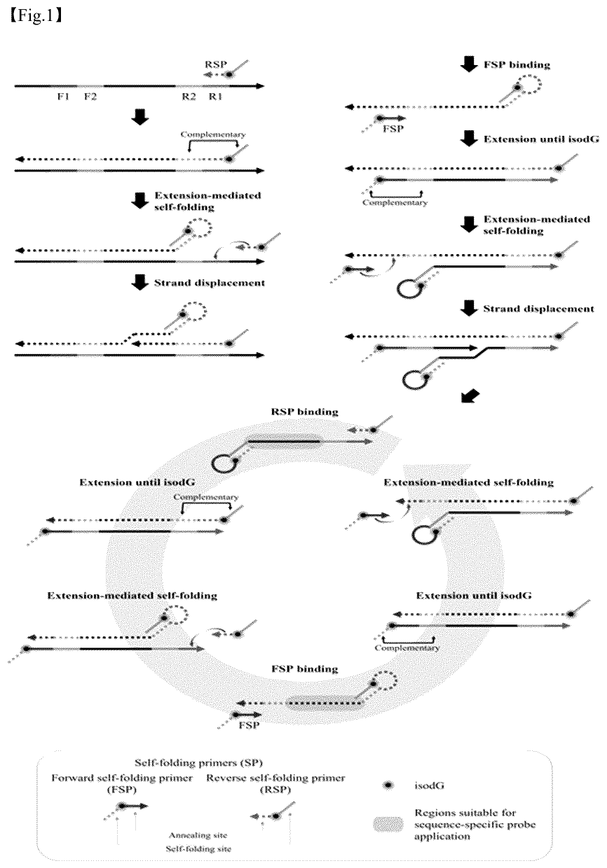

A schematic diagram illustrating an isothermal amplification mechanism using primers for extension-medicated self-folding isothermal amplification is shown in FIG. 1.

Furthermore, the present disclosure can provide a kit for extension-medicated self-folding isothermal amplification to detect a target nucleic acid, the kit including a primer set for extension-medicated self-folding isothermal amplification.

Herein, the term “target nucleic acid” may be any nucleic acid to be amplified, and the nucleic acid may be RNA or DNA. In addition, the target nucleic acid may be derived from a virus or a pathogenic microorganism, wherein the primer set may be used to detect the virus or pathogenic microorganism.

In one exemplary embodiment of the present disclosure, the SARS-CoV-2 virus gene was used as a target nucleic acid.

Since the kit of the present disclosure may be used in an isothermal amplification reaction, and unlike existing polymerase chain reaction (PCR) where denaturation, annealing, and extension reaction steps are carried out at different temperature conditions, the “isothermal amplification” refers to an amplification method requiring no separate special device since a nucleic acid amplification reaction is conducted at a constant temperature condition.

The kit may include, in addition to the primer set of the present disclosure, DNA polymerase, reaction buffers, dNTP, and MgCl2, for an amplification reaction.

Additionally, the primers of the present disclosure may further include a detection label to increase the convenience in detection. The detection label may be a compound, biomolecule, or biomolecule analog that is linked, bound, or attached to a primer to identify the density, concentration, or amount of an amplification product by typical methods. Examples thereof may include FAM, VIC, TET, JOE, HEX, CY3, CY5, ROX, RED610, TEXAS RED, RED670, TYE563, and NED.

Furthermore, the present disclosure can provide a method for isothermal amplification of a target nucleic acid by using a primer set for extension-mediated self-folding isothermal amplification, the method including amplifying a reaction mixture containing a target nucleic acid of a target subject and the primer set of the present disclosure under isothermal conditions.

The present inventors named the above-described method for isothermal amplification using the primer set developed in the present disclosure “extension-mediated self-folding Isothermal amplification technology (ExIT)”.

The extension-mediated self-folding Isothermal amplification technology according to the present disclosure, unlike existing isothermal amplification, corresponds to a method capable of exponential amplification of a target nucleic acid by using only the above-described one pair of primers developed in the present disclosure under the isothermal conditions.

The method for isothermal amplification of a target nucleic acid using the primer set for extension-mediated self-folding isothermal amplification according to the present disclosure may be performed as follows.

Specifically, the method for isothermal amplification of a target nucleic acid using the primer set for extension-mediated self-folding isothermal amplification according to the present disclosure is operated according to the following principles (see FIG. 1). The annealing site of the reverse primer binds to a target sequence complementary thereto in the nucleic acid sequence of a target subject and then extends. Since the extended sequence includes a sequence complementary to the folding site of the primer, self-folding occurs, and the target sequence within the target subject is again exposed and thus enables the binding with a new primer. A new reverse primer newly binds to the target sequence, and the nucleotide strand generated by the first reverse primer was released from the target subject owing to strand displacement activity of polymerase. Since the released ssDNA includes a sequence complementary to the annealing site of the forward primer, allowing the forward primer to bind thereto and extend. In such a case, the amplification reaction product does not contain an element capable of complementary reaction with isodG, and thus the polymerase stops its extension at the position of isodG. Thereafter, similar to the reverse primer, the forward primer underwent self-folding, and ssDNA is released due to strand replacement activity. The ssDNA generated by the forward primer enters the cycling pathway, and repeats procedures of the above-described “extension, self-folding, extension reaction stopping at an unnatural base, and release due to strand replacement activity”. As such, the ssDNA amplification product is repeatedly generated to obtain a large amount of target nucleic acid, which is recognized by the detection label (probe), enabling detection.

The reaction mixture for the reaction is prepared by mixing a target nucleic acid as a template for the amplification reaction and the primer set of the present disclosure designed on the basis of the nucleic acid, wherein the mixture is prepared by adding the heat-treated and subsequently cooled primer set to the target nucleic acid. Thereafter, an enzyme reaction solution containing DNA polymerase and a buffer is added to the mixture, followed by isothermal amplification at an isothermal amplification temperature.

The isothermal amplification temperature may be 60 to 65° C.

Additionally, the isothermal amplification reaction according to the present disclosure produces a reaction product in a single-stranded DNA (ssDNA).

The isothermal amplification method of the present disclosure enables the production of a reaction product in a single-stranded DNA state but not a double-stranded DNA state, unlike conventional isothermal amplification, and this is attributed to isodG inserted into the primer of the present disclosure.

Furthermore, the present disclosure can provide a method for detection of a target nucleic acid by using a primer set for extension-mediated self-folding isothermal amplification, the method including amplifying a specific target nucleic acid contained in a biological sample by using the primer set of the present disclosure under isothermal conditions.

The method for detection of a target nucleic acid of the present disclosure may be performed in the same procedure as in the above-described method for isothermal amplification of the present disclosure.

The method for detection of a target nucleic acid using a primer set for extension-mediated self-folding isothermal amplification of the present disclosure enables the detection of a nucleic acid through analysis of a target nucleic acid reaction product in a single-stranded DNA (ssDNA) state by isothermal amplification.

Additionally, the isothermal amplification may be conducted at a temperature of 60 to 65° C., and the target nucleic acid may be derived from a virus or pathogenic microorganism. One exemplary embodiment of the present disclosure confirmed that the SARS-CoV-2 virus gene could be accurately detected by the method of the present disclosure using the SARS-CoV-2 virus gene as a target nucleic acid.

According to one exemplary embodiment of the present disclosure, for the detection of the SARS-CoV-2 RNA virus N gene, the forward primer (5′-CCACGTTCCCGAisodGTTGGCATGGAAGTCACACCT-3′) and the reverse primer (5′-TGACGCATACAAisodGGCTCTGTTGGTGGGAATGTT-3′) were designed as a primer set for extension-mediated self-folding isothermal amplification, and as a result of performing an isothermal amplification reaction using the primer set, even a target nucleic acid present at a very low copy number, such as 90 copies, could be detected promptly and accurately, and only the target nucleic acid could be detected with high specificity.

As such, the isothermal amplification method and the target nucleic acid detection method each using a primer set for extension-mediated self-folding isothermal amplification of the present disclosure correspond to methods for one-step isothermal amplification of a nucleic acid and a signal probe by performing a reaction at a constant temperature using only one pair of primers, and the methods of the present disclosure require no special thermal conversion device, can achieve exponential amplification of a target nucleic acid promptly and conveniently, and can produce a large amount of single-stranded amplification product (ssDNA) by inducing self-folding after an extension reaction. Furthermore, the present disclosure can detect a target nucleic acid accurately and precisely, detect a target nucleic acid present at a low concentration with high reproducibility and precision, and achieve quantitative analysis.

### MODE FOR CARRYING OUT THE INVENTION

The following exemplary embodiments will be set forth for better understanding of the present disclosure. However, the following exemplary embodiments are merely provided to facilitate understanding of the present disclosure, and the scope of the present disclosure is not limited by the following exemplary embodiments.

### Preparative Example and Test Methods

### Chemicals and Reagents

All DNA samples used (see Table 1) were synthesized by Integrated DNA Technology (Skokie, IL, USA). SARS-CoV-2 RNA containing ORF, E, and, N genes were purchased from ATCC (Manassas, VA, USA). The expression plasmid SARS-CoV-2 N gene cDNA Clone (Native Sequence) was purchased from ORIGENE (Rockville, MD, USA). Human coronaviruses OC43, 229E, and NL63 were purchased from the Korea Bank for Pathogenic Viruses (Seoul, Korea). WarmStart® LAMP kit (DNA & RNA), Monarch® PCR & DNA Cleanup Kit, Monarch® DNA Gel Extraction Kit, Monarch® Total RNA Miniprep Kit, NEBNext® rRNA Depletion Kit v2 (Human/Mouse/Rat) with RNA Sample Purification Beads, and Deoxynucleotide (dNTP) Solution Mix, were purchased from New England Biolabs (Ipswich, MA, USA). Platinum™ SuperFi™ II DNA polymerase, ROX reference dye, and HotStart-IT binding proteins were purchased from Thermo Fisher Scientific (Waltham, MA, USA). All experiments were performed using UltraPure™ Distilled Water (Thermo Fisher Scientific) and TE (pH 8.0) (Bioneer®, Daejeon, Korea) to prevent DNA and RNA degradation.

TABLE 1

| | |
|---|---|
| Name | Sequence (5′→3′) |

polyT inserted ssDNA template

| | |
|---|---|
| polyT-0 | CGC GCA TTG GCA TGG AAG TCA CAC CTT CGG GAA CGT GGT TGA |
| | CCT ACA CAG GTG CCA TCA AAT TGGATG ACA AAG ATC CAA ATT TCA |
| | AAG ATC AAG TCA TTT TGC TGA ATA AGC ATA TTG ACG CAT ACA AAA |
| | CATTCC CAC CAA CAG AGC CTA AAA AGG ACA AAA AGA AGA AGG |
| | CTG ATG AAA CTC AAG CCT TAC CGCAG-C3 spacer |
| polyT-5 | CGC GCA TTG GCA TGG AAG TCA CAC CTT CGG GAA CGT GGT TGA |
| | CCT ACA CAG GTG CCA TCA AAT TGG ATG ACA AAG ATC CAA ATT |
| | TCA AAG ATC AAG TCA TTT TGC TGA ATA AGC ATA TTG ACG CAT ACA |
| | ATT TTT AAC ATT CCC ACC AAC AGA GCC TAA AAA GGA CAA AAA |
| | GAA GAA GGC TGA TGA AAC TCA AGC CTT AC-C3 spacer |
| polyT-10 | CGC GCA TTG GCA TGG AAG TCA CAC CTT CGG GAA CGT GGT TGA |
| | CCT ACA CAG GTG CCA TCA AAT TGG ATG ACA AAG ATC CAA ATT |
| | TCA AAG ATC AAG TCA TTT TGC TGA ATA AGC ATA TTG ACG CAT ACA |
| | ATT TTT TTT TTA ACA TTC CCA CCA ACA GAG CCT AAA AAG GAC |
| | AAAAAG AAG AAG GCT GAT GAA ACT CAA GC-C3 spacer |
| polyT-20 | CGC GCA TTG GCA TGG AAG TCA CAC CTT CGG GAA CGT GGT TGA |
| | CCT ACA CAG GTG CCA TCA AAT TGG ATG ACA AAG ATC CAA ATT |
| | TCA AAG ATC AAG TCA TTT TGC TGA ATA AGC ATA TTG ACG CAT ACA |
| | ATT TTT TTT TTT TTT TTT TTT AAC ATT CCC ACC AAC AGA GCC TAA |
| | AAA GGA CAA AAA GAA GAA GGC TGA TG-C3 spacer |

dsDNA template sequence

| | |
|---|---|
| dsDNA template | CAC TCA ACA TGG CAA GGA AGA CCT TAA ATT CCC TCG AGG ACA |
| (168-1184, 996 bp) | AGG CGT TCC AAT TAA CAC CAA TAG CAG TCC AGA TGA CCA AAT |
| | TGG CTA CTA CCG AAG AGC TAC CAG ACG AAT TCG TGG TGG TGA |
| | CGG TAA AAT GAA AGA TCT CAG TCC AAG ATG GTA TTT CTA CTA CCT |
| | AGG AAC TGG GCC AGA AGC TGG ACT TCC CTA TGG TGC TAA CAA |
| | AGA CGG CAT CAT ATG GGT TGC AAC TGA GGG AGC CTT GAA TAC |
| | ACC AAA AGA TCA CAT TGG CAC CCG CAA TCC TGC TAA CAA TGC |
| | TGC AAT CGT GCT ACA ACT TCC TCA AGG AAC AAC ATT GCC AAA |
| | AGG CTT CTA CGC AGA AGG GAG CAG AGG CGG CAG TCA AGC CTC |
| | TTC TCG TTC CTC ATC ACG TAG TCG CAA CAG TTC AAG AAA TTC |
| | AAC TCC AGG CAG CAG TAG GGG AAC TTC TCC TGC TAG AAT GGC |
| | TGG CAA TGG CGG TGA TGC TGC TCT TGC TTT GCT GCT GCT TGA |
| | CAG ATT GAA CCA GCT TGA GAG CAA AAT GTC TGG TAA AGG CCA |
| | ACA ACA ACA AGG CCA AAC TGT CAC TAA GAA ATC TGC TGC TGA |
| | GGC TTC TAA GAA GCC TCG GCA AAA ACG TAC TGC CAC TAA AGC |
| | ATA CAA TGT AAC ACA AGC TTT CGG CAG ACG TGG TCC AGA ACA |
| | AAC CCA AGG AAA TTT TGG GGA CCA GGA ACT AAT CAG ACA AGG |
| | AAC TGA TTA CAA ACA TTG GCC GCA AAT TGC ACA ATT TGC CCC |
| | CAG CGC TTC AGC GTT CTT CGG AAT GTC GCG CAT TGG CAT GGA |
| | AGT CAC ACC TTC GGG AAC GTG GTT GAC CTA CAC AGG TGC CAT |
| | CAA ATT GGA TGA CAA AGA TCC AAA TTT CAA AGA TCA AGT CAT |
| | TTT GCT GAA TAA GCA TAT TGA CGC ATA CAA AAC ATT CCC ACC AAC |
| | AGA GCC TAA AAA GGA CAA AAA GAA GAA GGC TGA TGA AAC TCA |
| | AGC CTT ACC GCA GAG ACA GAA GAA |

Self-folding primers*

| | |
|---|---|
| FSP (0-21 nt) | GTA+ GGT+ CAA+ CGT+ TCC+ CGA+ TTG GCA TTG GCA TGG AAG TCA |
| | CAC CT |
| RSP (0-21 nt) | TAA+ GCA+ TAT+ TGA+ CGC+ ATA+ CAA+ GCT CTG TTG GTG GGA ATG |
| | TT |
| FSP without isodG | CCA CGT TCC CGA TT GGC ATG GAA GTC ACA CCT |
| RSP without isodG | TGA CGC ATA CAA GC TCT GTT GGT GGG AAT GTT |
| FSP | CCA CGT TCC CGA-isodG-TT GGC ATG GAA GTC ACA CCT |
| RSP | TGA CGC ATA CAA-isodG-GC TCT GTT GGT GGG AAT GTT |

Probes

| | |
|---|---|
| MB (FSP) | 6-FAM-TC AGC AAA ATG ACT TGA TCT TTG AAA TTT CAT TTT GCT GA- |
| | DABCYL |
| MB (RSP) | 6-FAM-TC AGC AAA ATG AAA TTT CAA AGA TCA AGT CAT TTT GCT |
| | GA-DABCYL |
| MB (FSP) | AAA TTT CAA AGA TCA AGT CAT TTT GCT GA |
| complementary sequence | |
| MB (RSP) | TCA GCA AAA TGA CTT GAT CTT TGA AAT TT |
| complementary sequence | |

*The colors of oligonucleotide sequences correspond to those of the domains depicted in FIG. 1. The red and blue colors indicate the annealing site of forward self-folding primer (FSP) and reverse self-folding primer (RSP), respectively. The orange and green colors represent the self-folding site of FSP and RSP, respectively.

### Feasibility and Optimization of ExIT

### (1) Self-Folding Primer Design

The self-folding primer (SP) was selected and designed based on the recently available full sequence of SARS CoV-2 from the NCBI Reference Sequence Database (https://www.ncbi.nlm.nih.gov/nuccore/NC_045512.2). An annealing site for reverse SP (RSP) was taken directly from a reverse primer, and the NCBI Primer BLAST (https://www.ncbi.nlm.nih.gov/tools/primer-blast/) was used to design an annealing site for forward SP (FSP).

### (2) Efficiency of Self-Folding According to the Distance Between Detection Sites

To verify self-folding efficiency at the detection site, a synthesized ssDNA template containing various numbers of poly T molecules between the detection sites was used. The experiment was conducted in a final volume of 25 μL solution containing 1.6 μM RSP (21 nt), 50 nM MB (RSP), 1× WarmStart LAMP Mater Mix, 1× ROX reference dye with 1×10⁹ copies of ssDNA template inserted poly T (0, 5, 10, 20 nt) at 62° C. for 120 min. Before the experiment, 500 nM of MB (FSP) or MB (RSP) in 1× WarmStart LAMP Master Mix was heated up to 95° C. for 5 min, followed by slow cooling down to 20° C.

(3) Selection of Primer Set with Highest Amplification Efficiency

The SARS-CoV-2 N gene cDNA clone was amplified to obtain dsDNA templates. Amplicons were extracted and purified according to the manufacturer's instructions. The purified products were measured by using a DS-11 FX+ spectrophotometer using a Denovix dsDNA High Sensitivity Fluorescent Assay kit (Denovix, DE, USA), and stored at −20° C. The target dsDNA was heated at 95° C. 5 min before use.

To optimize the length of SP, the reaction mixture contained 1×10⁹ copies of dsDNA template, 50 nM (RSP or FSP), 1× WarmStart LAMP Master Mix, 1× ROX reference dye with various lengths of the self-folding site of SP (0, 3, 6, 9, 12, 15, 18, or 21 nt) at 60, 62, and 64° C. A group without the addition of the dsDNA template was used as the negative control.

To determine a primer set with the best self-folding efficiency, amplification was performed using primers selected for each temperature. The experiment to identify the best primer set was conducted in a final volume of 25 μL reaction solution containing 1.6 μM FSP (12, 15, or 21 nt), 1.6 μM RSP (12 or 21 nt), 1× ROX reference dye, 50 nM MB (RSP), and 0-9×10² copies of SARS-CoV-2 RNA (ATCC, VR-3276SD) in 1× WarmStart LAMP Master Mix at 60, 62, and 64° C.

### Sensitivity of ExIT

The sensitivity of ExIT was assayed in a final volume of 25 μL solution containing 1.6 μM FSP, 1.6 μM RSP, 1× ROX reference dye, 50 nM MB (RSP), and target RNA at various concentrations (0-9×10⁴ copies of SARS-CoV-2 RNA) contained in 1× WarmStart LAMP Master Mix. The prepared reaction solution was incubated at 60° C. for 90 min by StepOnePlus™ Real-Time PCR System.

### Specificity of ExIT

Human total RNA was obtained from HEK cells, purified using Monarch® Total RNA Miniprep Kit, and depleted using NEBNext® rRNA Depletion Kit v2 (Human/Mouse/Rat) with RNA Sample Purification Beads according to the manufacturer's protocol. The final products were measured by using a DS-11 FX+ spectrophotometer using a Denovix RNA Quantification Assay (Denovix, DE, USA), and stored at −80° C. before use.

ExIT specific for the SARS-CoV-2 N gene was performed using various templates (NTC, human total RNA, human coronavirus 229E, human coronavirus OC43, and human coronavirus NL63) in the absence or presence of SARS-CoV-2 N. Each sample was then analyzed using the reaction procedure described above. The concentration of SARS-CoV-2 and human coronaviruses (OC43, 229E, and NL63) was 9×10⁴ copies/μL. Total human RNA obtained from HEK cells was 1 ng.

### Example 1

### Primer Design

### <1-1> Self-Folding Primer (SP) Design

The present inventors designed self-folding primers as shown in Table 2 below. An annealing site for reverse SP (hereinafter, RSP) was taken directly from a reverse primer targeting N gene in Park, M. et al. (Exp Mol Med 52, 963-977 (2020)). An annealing site of the forward SP (hereinafter, FSP), which has Tm at the equivalent level to the annealing site of the previously selected reverse primer, was selected using NCBI Primer-Blast Tool program. The sequences of the annealing sites of SP are shown in column 5 of Table 2 below.

A sequence complementary to a product formed by extension from the annealing site is connected to the 5′-end of the annealing site and is selected as a folding site, and for the optimization of the length of the folding site, the self-folding site was designed with various lengths at intervals of 3 nt, and the sequences of the folding sites of SP are shown in column 3 of Table 2 below.

TABLE 2
Self-folding primer sequences

| | | SP sequence (5′→3′) | | |
|---|---|---|---|---|
| | | Self-folding | | Annealing |
| Name | Direction | site | Modification | site |
| (1) FSP | Forward | GTA+GGT+CAA+CCA | — | TTGGCATGG |
| (0-21 nt) | | +CGT+TCC+CGA | | AAGTCACAC |
| | | | | CT |
| (2) RSP | Reverse | TAA+GCA+TAT+TGA | — | GCTCTGTTG |
| (0-21 nt) | | +CGC+ATA+CAA | | GTGGGAATG |
| | | | | TT |
| (3) FSP | Forward | CCACGTTCCCGA | — | TTGGCATGG |
| without | | | | AAGTCACAC |
| isodG | | | | CT |
| (4) RSP | Reverse | TGACGCATACAA | — | GCTCTGTTG |
| without | | | | GTGGGAATG |
| isodG | | | | TT |
| (5) FSP | Forward | CCACGTTCCCGA | isodG | TTGGCATGG |
| | | | | AAGTCACAC |
| | | | | CT |
| (6) RSP | Reverse | TGACGCATACAA | isodG | GCTCTGTTG |
| | | | | GIGGGAATG |
| | | | | TT |

<1-2> Design of SP Having isodG Inserted Therein

The optimal lengths of folding sites of SP were selected through experiments, and the corresponding sequences are shown on lines 3 and 4 of Table 2 above. In addition, isodG was inserted between the folding site and the annealing site, and the sequences of SP having isodG inserted therein (hereinafter, iSP) are shown on lines 5 and 6 of Table 2 above.

### Example 2

Conditions of Self-Folding Primers for Isothermal Amplification Reaction with Excellent Detection Performance

### <2-1> Efficiency of Self-Folding According to Removal of Distance Between Target Recognition Sequences

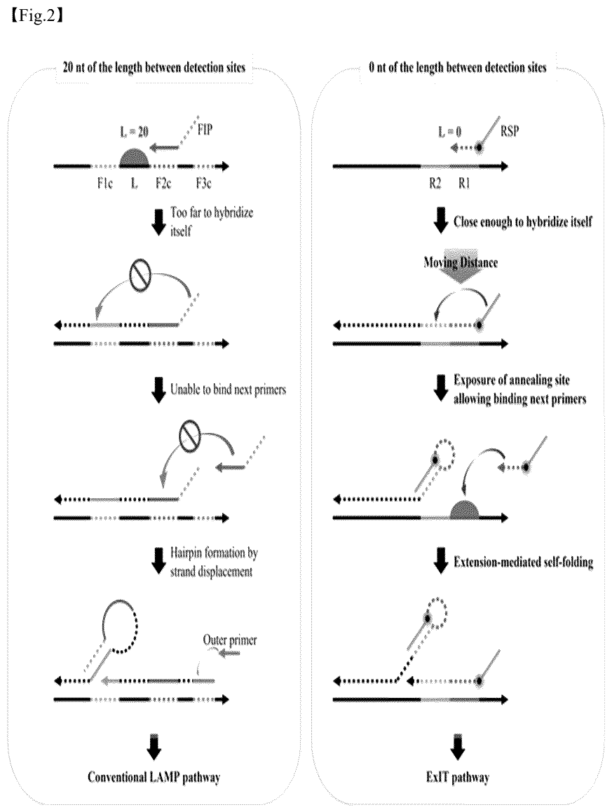

In LAMP, the distance between target recognition sequences is about 20 nt. If the inner primer binds to F2c and extends to F1c, the distance reaches approximately 60 nt, and thus it is difficult for the inner primer to unwind the double-stranded region to configure a hairpin. Moreover, the annealing site (F2c) is not exposed, and thus the hairpin formation requires strand replacement activity of the outer primer, which can lead to a step for an intermediate product. Therefore, at least four primers including the inner and outer primer sets are required. Hence, the present inventors assumed that when the distance between target recognition sequences is removed, the distance between R1 and R2 is also narrowed, and R2 within RSP meets with R2c generated after the extension of RSP, thereby increasing the likelihood of forming a folding structure. Once the self-folding occurs, the annealing site (R1) is again exposed, which may lead to a next step without the outer primer. Therefore, the following experiment was conducted to investigate the assumption. The ssDNA templates were prepared by inserting polyT sequences of 0, 5, 10, or 20 nt between the target sequences, and the degree of linear amplification efficiency was compared using RSP (21 nt) similar to the length of the inner primer of LAMP. An experiment was conducted by the above-described self-folding efficiency analysis method according to the distance between the detection sites. A comparative schematic diagram of the self-folding mechanisms for different distances between target recognition sites is shown in FIG. 2.

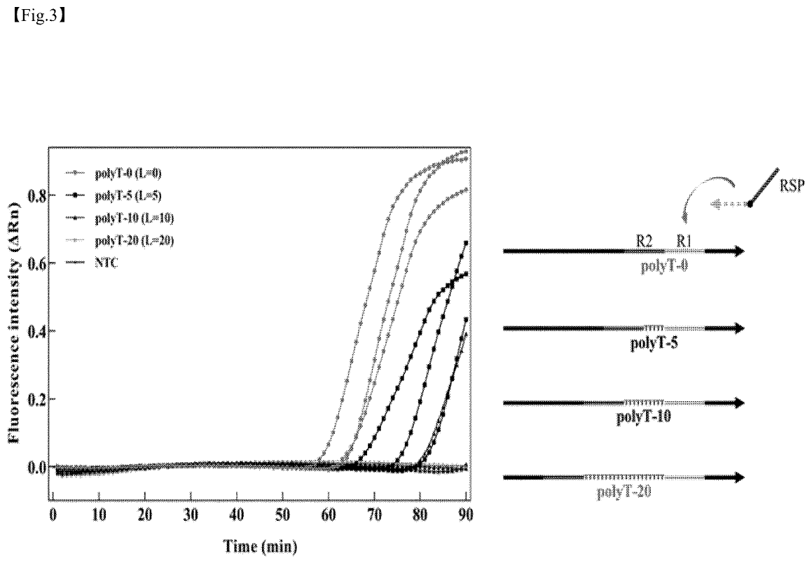

As a result of analysis, as shown in FIG. 3, the fluorescence signal appeared more quickly as the distance between target recognition sites was decreased. Particularly, the fluorescence signal appeared most quickly when poly-T of 0 nt was used, and this corresponded to a group where the distance between target recognition sites was removed. It was therefore seen that the removal of the distance between target recognition sequences can improve linear amplification efficiency due to self-folding.

<2-2> Length and Amplification Conditions of Self-Folding Primers with Highest Amplification Efficiency

The present inventors investigated optimum amplification conditions considering the following two factors. First, the self-folding efficiency according to the length of the self-folding site of each primer was analyzed. This affects the ability to maintain the folding structure after primer extension. If the folding site of the SP is too short, the folding structure is difficult to maintain, but if the length is too long, there is a higher likelihood of dimer formation between primers. Therefore, the length of the primer suitable for amplification was investigated. Second, the self-folding efficiency for each primer length according to the temperature was analyzed. If the amplification is performed at a temperature higher than the Tm of the folding structure, the folding structure may not be maintained and may unravel. Therefore, it is necessary to determine the appropriate temperature for each folding site length. Specifically, the analysis was performed by the above-described method for selecting a primer set with highest amplification efficiency.

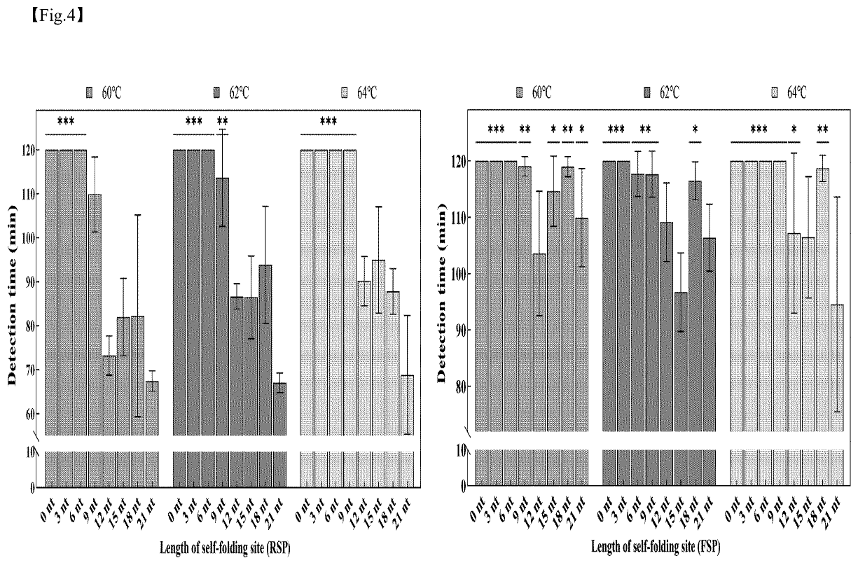

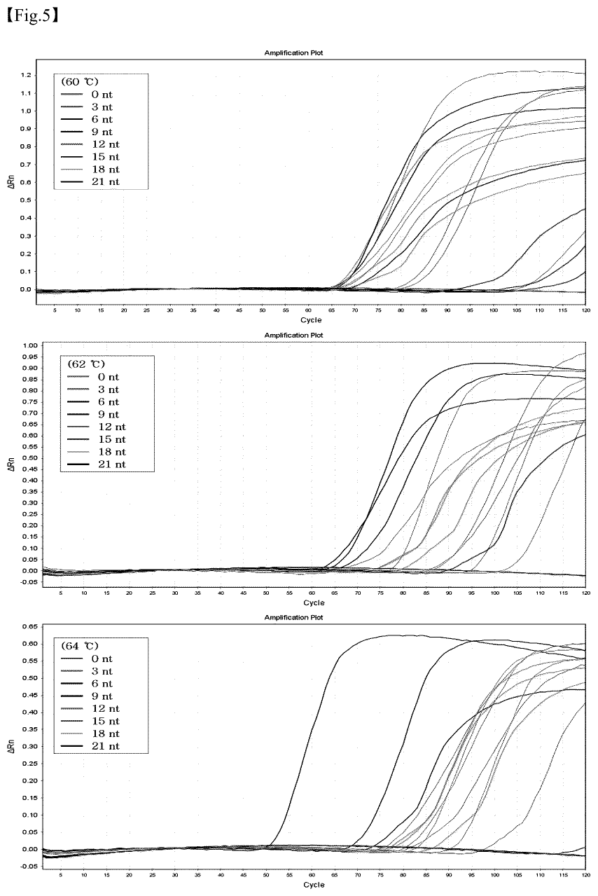

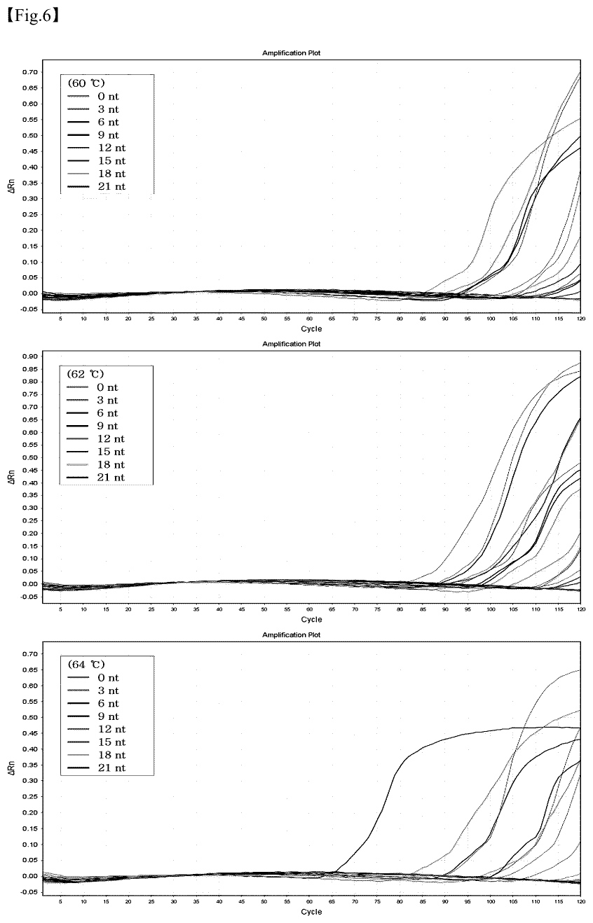

As a result, as shown in FIGS. 4 to 6, for RSP, when the length of the self-folding site was 12 nt or 21 nt, a fastest detection time was observed at 60° C. At both 62° C. and 64° C., 21 nt was found to show a fastest detection time. For FSP, 12 nt at 60° C., 15 nt at 62° C., and 21 nt at 64° C. were found to show fastest detection times. Through these results, the present inventors selected primer candidates with a self-folding site showing a fastest detection time, and the selected primer candidates showing a fastest detection time at the same temperature were combined, and used to investigate the detection sensitivity and amplification efficiency of SARS-CoV-2 RNA. The selected primer candidates are shown in Table 3.

TABLE 3
Selected primer candidates and comparison of detection limits thereof

| Selected | SP length | Detection time (min) | | | | | | | | | | |
|---|---|---|---|---|---|---|---|---|---|---|---|---|
| candidate | and | 0 | | | 90 | | | Average | 900 | | | Average |
| set | temperature | copies | | | copies | | | (RSD) | copies | | | (RSD) |
| 1 | FSP (12 nt) + | — | — | — | 55.0 | 38.3 | 34.1 | 42.5 | 36.6 | 36.0 | 36.0 | 36.2 |
| | RSP (12 nt) | | | | | | | (11.05) | | | | (0.34) |
| | at 60° C. | | | | | | | | | | | |
| 2 | FSP (12 nt) + | — | — | — | 49.0 | — | — | — | 41.0 | 40.5 | 41.2 | 40.9 |
| | RSP (21 nt) | | | | | | | | | | | (0.36) |
| | at 60° C. | | | | | | | | | | | |
| 3 | FSP (15 nt) + | — | — | — | 39.0 | — | 40.3 | — | 40.1 | 38.0 | 45.6 | 41.2 |
| | RSP (21 nt) | | | | | | | | | | | (3.92) |
| | at 62° C. | | | | | | | | | | | |
| 4 | FSP (21 nt) + | — | — | — | — | 43.0 | 39.4 | — | 37.3 | 45.0 | 46.9 | 43.1 |
| | RSP (21 nt) | | | | | | | | | | | (5.08) |
| | at 64° C. | | | | | | | | | | | |

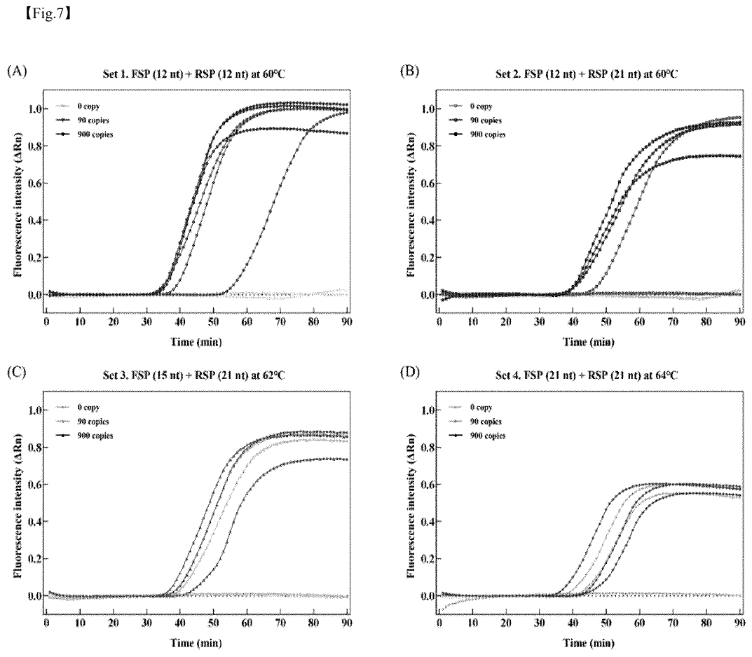

As a result of analyzing the detection sensitivity and detection limit of SARS-CoV-2 RNA by using primers of four candidate sets, as shown in Table 3 and FIG. 7, a fluorescence signal was observed from all the candidate sets when 900 copies of SARS-CoV-2 RNA were present. Especially, Candidate 1 showed a fastest detection time of 36.2 min, with a smallest variation. However, when SARS-CoV-2 RNA was decreased to 90 copies, only Candidate 1 showed uniform amplification. It was seen through these results that among the four candidate sets of primers on Table 3, the primer set of Candidate 1 showed the best detection performance. Thus, the primer set of Candidate 1 was used for the following experiment.

### Example 3

Detection Performance of SARS-CoV-2 RNA Virus N Gene Using Self-Folding Primers Having isodG Inserted Therein

### <3-1> Detection Sensitivity

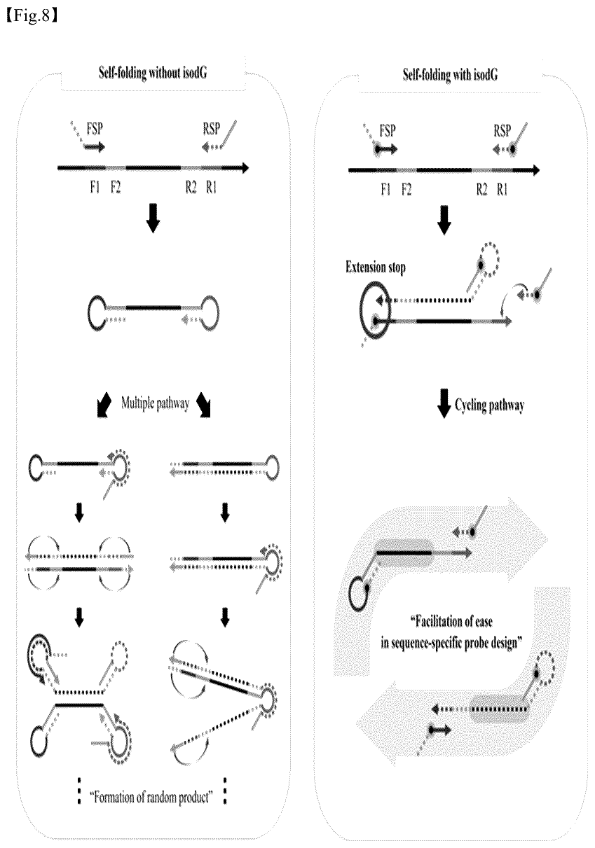

To improve the sensitivity and reproducibility of the self-folding primers (SP) of the present disclosure, the present inventors inserted isodG into the self-folding primers to induce a cycling pathway for forming an ssDNA amplification product. As for SP without isodG, polymerase operates to the end of SP, and an intermediate product is formed like in LAMP. If the extension of the intermediate product continues or another SP binds to the loop site, various forms of dsDNA amplification products may be formed, and in such a case, ssDNA amplification products, to which a probe is to be introduced, are not sufficiently generated, resulting in poor sensitivity and reproducibility. However, in cases of using SP having isodG inserted therein, developed in the present disclosure, the polymerase stops its extension at the position of isodG, and the amplification product remains in an ssDNA state, and an intermediate product may also be maintained in an ssDNA state. Through repeated binding and extension of SP with isodG, the SP enters the cycling pathway, thereby forming a large amount of ssDNA amplification products. In such a case, the probe design and introduction utilizing the ssDNA amplification product is easy, leading to excellent sensitivity and reproducibility. As described above, a comparative schematic diagram of mechanisms of reaction amplification product formation in the presence and absence of isodG is shown in FIG. 8.

The present inventors analyzed the detection sensitivity of the SARS-CoV-2 RNA virus N gene by using self-folding primers with or without isodG, and to achieve this, the sensitivity of the above-described ExIT was analyzed. Specifically, a comparative analysis was performed on Candidate Set 1 on Table 3 by using one set having isodG inserted therein and one set without isodG.

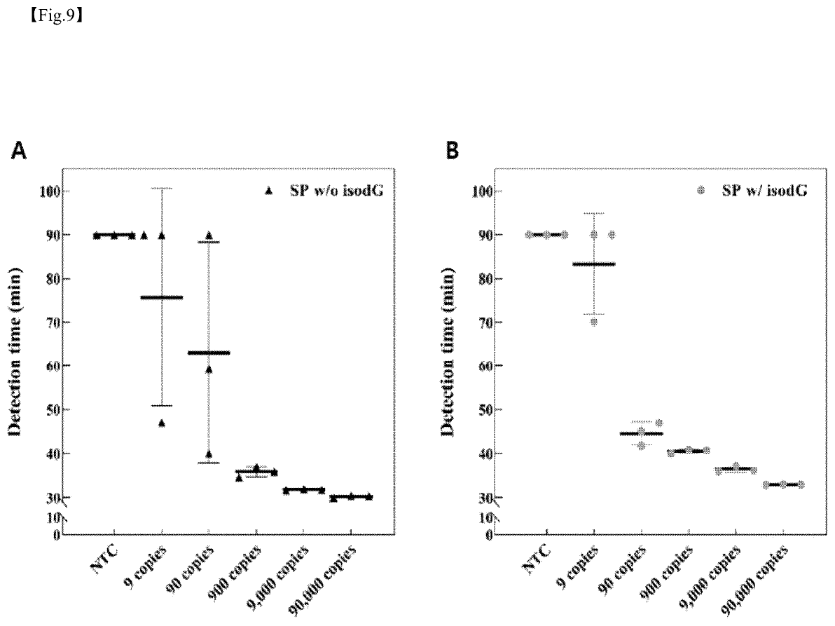

As a result, as shown in FIG. 9, as a result of investigating the sensitivity of SARS-CoV-2 RNA by SP without isodG, signals were detected uniformly up to 900 copies, but below 90 copies, only some samples showed signals, with an increase in deviation observed. Additionally, when summarizing with the previous experimental results on Candidate 1 in order to select SP combinations, a consistent reproducibility was not observed below 900 copies. This was because for SP without isodG, the recognition rate of the probe was lowered due to randomly generated dsDNA products.

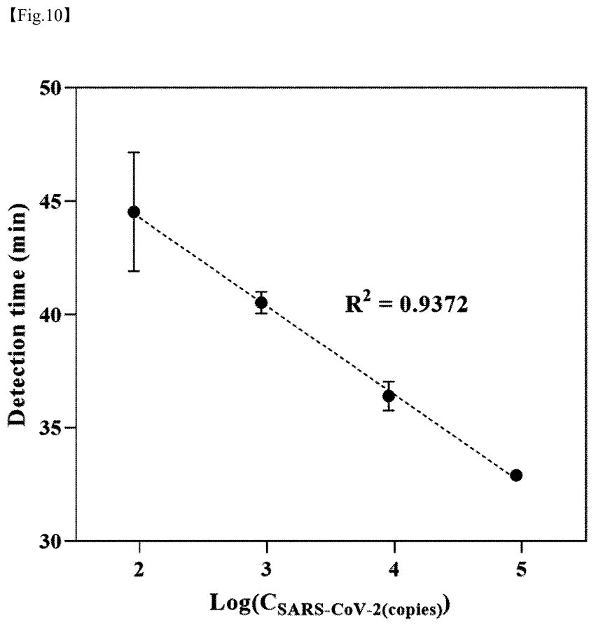

However, as a result of investigating the sensitivity of SARS-CoV-2 RNA by using SP containing isodG, signals were uniformly detected even for 90 copies of RNA (FIG. 9B). Furthermore, during the measurement of the sensitivity by using SP containing isodG, the relationship between the log value of a specific concentration range (90 copies to 90,000 copies of RNA) showing uniform signals and the detection time was investigated. As a result, as shown in FIG. 10, the correlation coefficient (R2) was 0.9372, indicating high linearity. That is, the insertion of isodG into the self-folding primers can stably produce ssDNA amplification products and can ensure increased detection reproducibility and improved sensitivity.

### <3-2> Detection Specificity

Next, to investigate the detection specificity of the ExIT developed in the present disclosure, analysis was performed using various coronaviruses and human total RNA. First, human coronaviruses OC43, 229E, and NL63, which belong to the same family Coronaviridae as SARS-CoV-2. Out of these, OC43 belongs to the same genus Betacoronavirus as SARS-CoV-2. In addition, for use in the disease diagnosis of actual patients, human total RNA was included in a control group. The specificity of the ExIT was investigated according to the presence or absence of the target SARS-CoV-2 RNA, and the experiment was conducted by the above-described method for analyzing the detection specificity of ExIT.

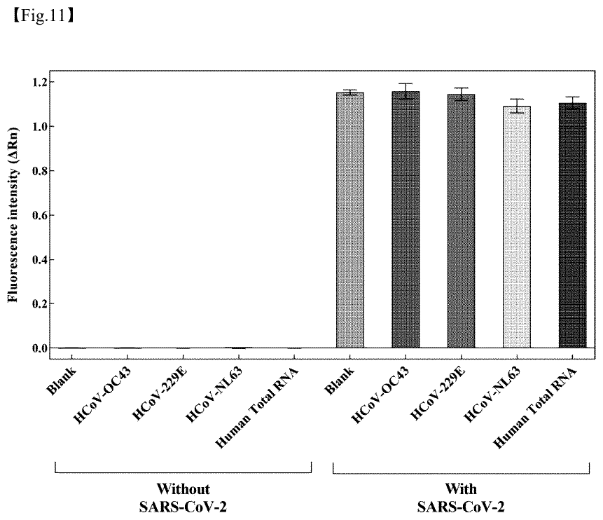

As a result, as shown in FIG. 11, when the extension-mediated self-folding isothermal amplification technology (ExIT) using the self-folding primers of the present disclosure was performed, no signal was detected in the experimental group without SARS-CoV-2 RNA. This indicates that even if there were unintended interactions between non-specific samples and the self-folding primers of the present disclosure, they never affected the detection. Furthermore, even the presence of non-complementary samples did not affect the detection of SARS-CoV-2, and the target nucleic acid SARS-CoV-2 gene could be detected with high specificity.

The above results identified that the extension-mediated self-folding isothermal amplification technology using the self-folding primers developed in the present disclosure enables exponential amplification under isothermal conditions by using only one pair of primers, as well as the production of ssDNA reaction products, thereby enabling detection of sequence-specific target gene (nucleic acid) as well as accurate detection with high sensitivity and specificity.

As set forth above, the present disclosure has been described with reference to preferable examples. A person skilled in the art to which the present disclosure pertain would understand that the present disclosure could be implemented in a modified form without departing from the inherent characteristics of the present disclosure. Accordingly, the exemplary embodiments described herein should be considered from an illustrative aspect rather than from a restrictive aspect. The scope of the present disclosure should be defined not by the detailed description but by the appended claims, and all differences falling within a scope equivalent to the claims should be construed as being included in the present disclosure.

1. A primer set for extension-mediated self-folding isothermal amplification, the primer set comprising a pair of primers consisting of a forward primer and a reverse primer,
wherein the 3′-region of each of the primers includes an annealing site consisting of a 15- to 25-nucleotide sequence complementary to a target sequence of a target subject,
the 5′-region of each of the primers includes a self-folding site consisting of a 10- to 25-nucleotide sequence complementary to a nucleic acid sequence of an amplification reaction product to form a hairpin structure, and
isodG, an unnatural base, is inserted between the annealing site and the self-folding site.

2. The primer set of claim 1, wherein the unnatural base is one isodG that is inserted.

3. The primer set of claim 1, wherein the self-folding site consists of a 12- to 21-nucleotide sequence complementary to the nucleic acid sequence of the amplification reaction product to form a stable hairpin structure.

4. A kit for extension-mediated self-folding isothermal amplification to detect a target nucleic acid, the kit comprising the primer set of claim 1.

5. The kit of claim 4, wherein the target nucleic acid is derived from a virus or a pathogenic microorganism.

6. A method for isothermal amplification of a target nucleic acid by using a primer set for extension-mediated self-folding isothermal amplification, the method comprising subjecting a reaction mixture, containing a target nucleic acid of a target subject and the primer set of claim 1, to isothermal amplification.

7. The method of claim 6, wherein a reaction product in a single-stranded DNA (ssDNA) state is produced by the isothermal amplification.

8. The method of claim 6, wherein the isothermal amplification is performed at a temperature of 60° C. to 65° C.

9. A method for detection of a target nucleic acid by using a primer set for extension-mediated self-folding isothermal amplification, the method comprising subjecting a specific target nucleic acid, contained in a biological sample, to isothermal amplification by using the primer set of claim 1.

10. The method of claim 9, wherein a target nucleic acid reaction product in a single-stranded DNA (ssDNA) state is produced by the isothermal amplification.

11. The method of claim 9, wherein the isothermal amplification is performed at a temperature of 60° C. to 65° C.

12. The method of claim 9, wherein the target nucleic acid is derived from a virus or a pathogenic microorganism.
